(19) United States
(12) Patent Application Publication
(10) Pub. No.: US 2011/0206643 A1
(43) Pub. Date: Aug. 25, 2011

(54) Automated Cell Therapy System

(75) Inventors: Valentin Fulga, Tel Aviv (IL); Robert Michael Clark, Bangkok (TH); Yael Porat, Hod Hasharon (IL); Danny Belkin, Givat Shmuel, IL (US); Avner Schrift, Rishon Lezion (IL); Eyal Teichman, Had Hasharon (IL)

(73) Assignees: IN MOTION INVESTMENT, LTD., Kowloon (HK); Kwalata Trading Limited

(21) Appl. No.: 11/921,546

(22) PCT Filed: May 31, 2006
(86) PCT No.: PCT/IL2006/000633

§ 371 Date: Mar. 1, 2011

Related U.S. Application Data
(60) Provisional application No. 60/687,115, filed on Jun. 2, 2005.

(51) Int. Cl. A61K 35/12; C12M 3/00; C12M 1/36; C12N 5/07; C12Q 3/00

(52) U.S. Cl. 424/93.7; 435/289.1; 435/286.1; 435/305.1; 435/325; 435/3

(57) ABSTRACT

A method for automated cell processing is provided, including receiving at least one tissue containing a multiplicity of cells belonging to multiple cell types, and automatically increasing both the proportion of cells of at least one of said multiple cell types as compared with at least another of said multiple cell types and the absolute number of cells of said at least one of said multiple cell types. Other embodiments are also described.

### CROSS REFERENCES TO RELATED APPLICATIONS

The present application claims the benefit of U.S. Provisional Patent Application 60/687,115 to Belkin et al., filed Jun. 2, 2005, and is related to U.S. Provisional Patent Application Ser. No. 60/576,266 filed Jun. 1, 2004 and entitled IN VITRO TECHNIQUES FOR USE WITH STEM CELLS, to U.S. Provisional Patent Application Ser. No. 60/588,520 filed Jul. 15, 2004 and entitled INDICATIONS FOR STEM CELL USE, to U.S. Provisional Patent Application Ser. No. 60/631,098 filed Nov. 24, 2004 and entitled A METHOD TO ACCELERATE STEM CELL RECRUITMENT AND HOMING and to U.S. Provisional Patent Application Ser. No. 60/636,391 filed Dec. 14, 2004 and entitled REGULATING STEM CELLS. Each of the above-referenced applications is hereby incorporated by reference.

### FIELD OF THE INVENTION

The present invention relates to cell culturing systems generally, and specifically to automated cell culturing systems for cell therapy, diagnostic testing and biological research and development.

### BACKGROUND OF THE INVENTION

The following U.S. published patent documents, which are incorporated herein by reference, are believed to represent the current state of the art:

U.S. Pat. Nos. 5,424,209; 5,473,706; 4,966,853; 4,090,921; 6,790,654; 6,673,595; 6,261,832; 6,238,908; 6,228,635; 6,048,721; 6,096,532; 6,066,497; 4,696,902; 6,780,617; 6,127,141 and 5,985,653.

### SUMMARY OF THE INVENTION

Some embodiments of the present invention seek to provide an automated cell culturing system designed for culturing cells, which are useful in cell therapy, diagnostic testing and biological research and development.

There is thus provided in accordance with an embodiment of the present invention an automated cell processing system including functionality which receives at least one tissue containing a multiplicity of cells belonging to multiple cell types and automatic functionality which modulates (e.g., increases or decreases) both the proportion of cells of at least one of the multiple cell types as compared with at least another of the multiple cell types and also modulates (e.g., increases or decreases) the absolute number of cells of the at least one of the multiple cell types.

There is also provided in accordance with another embodiment of the present invention an automated cell processing system including functionality which receives at least one tissue containing a multiplicity of cells belonging to multiple cell types and automatic functionality including cell culturing which modulates (e.g., increases or decreases) the proportion of cells of at least one of the multiple cell types as compared with at least another of the multiple cell types.

There is further provided in accordance with a further embodiment of the present invention an automated cell processing system including a first functionality which receives at least one tissue containing a first plurality of cell types having a first proportionality of numbers, at least one second functionality operative on at least some of the first plurality of cell types and adaptive control functionality operative to control the operation of the at least one second functionality as a function of at least some of the numbers.

There is additionally provided in accordance with another embodiment of the present invention an automated cell processing system including a first functionality which receives at least one tissue containing a first plurality of cell types having a first proportionality of numbers, at least one second functionality operative on a second plurality of cell types being at least some of the first plurality of cell types and having a second proportionality of numbers which varies over time, monitoring functionality operative to monitor variations in the numbers of the second plurality of cell types and adaptive control functionality operative to control the operation of the at least one second functionality as a function of at least some of the variations in the numbers of the second plurality of cell types.

There is still further provided in accordance with a further embodiment of the present invention an automated cell processing system including a first functionality which receives at least one tissue containing a first plurality of cell types having a first proportionality of numbers, at least one second functionality operative on at least some of the first plurality of cell types, monitoring functionality operative to monitor differentiation of the at least some of the first plurality of cell types and adaptive control functionality operative to control the operation of the at least one second functionality in response to at least one output of the monitoring functionality.

There is additionally provided in accordance with yet a further embodiment of the present invention an automated cell processing system including a first functionality which receives at least one tissue containing a first plurality of cell types having a first proportionality of numbers, at least one second functionality operative with respect to at least some of the first plurality of cell types to at least one of add, mix or remove at least one material, monitoring functionality operative to monitor the at least some of the first plurality of cell types and adaptive control functionality operative to control the operation of the at least one second functionality in response to at least one output of the monitoring functionality.

There is also provided in accordance with another embodiment of the present invention an automated cell bank generating system including functionality which receives at least one tissue containing a multiplicity of cells belonging to multiple cell types, automatic functionality which modulates (e.g., increases or decreases) both the proportion of cells of at least one of the multiple cell types as compared with at least another of the multiple cell types and also modulates (e.g., increases or decreases) the absolute number of cells of the at least one of the multiple cell types and storage functionality for storing the at least one of the multiple cell types.

There is additionally provided in accordance with an additional embodiment of the present invention an automated cell bank generating system including functionality which receives at least one tissue containing a multiplicity of cells belonging to multiple cell types, automatic functionality including cell culturing which modulates (e.g., increases or decreases) the proportion of cells of at least one of the multiple cell types as compared with at least another of the multiple cell types and storage functionality for storing the at least one of the multiple cell types.

There is further provided in accordance with a further embodiment of the present invention an automated cell bank generating system including a first functionality which receives at least one tissue containing a first plurality of cell types having a first proportionality of numbers, at least one second functionality operative on at least some of the first plurality of cell types, adaptive control functionality operative to control the operation of the at least one second functionality as a function of at least some of the numbers and storage functionality for storing the at least some of the first plurality of cell types.

There is also provided in accordance with an embodiment of the present invention an automated long-term cell monitoring system including functionality which receives at least one tissue containing a multiplicity of cells belonging to multiple cell types, automatic functionality which modulates (e.g., increases or decreases) both the proportion of cells of at least one of the multiple cell types as compared with at least another of the multiple cell types and also modulates (e.g., increases or decreases) the absolute number of cells of the at least one of the multiple cell types, and long-term monitored culturing functionality for storing the at least one of the multiple cell types.

There is additionally provided in accordance with an embodiment of the present invention an automated long-term cell monitoring system including functionality which receives at least one tissue containing a multiplicity of cells belonging to multiple cell types, automatic functionality including cell culturing which modulates (e.g., increases or decreases) the proportion of cells of at least one of the multiple cell types as compared with at least another of the multiple cell types, and long-term monitored culturing functionality for storing the at least one of the multiple cell types.

There is further provided in accordance with an embodiment of the present invention an automated long-term cell monitoring system including a first functionality which receives at least one tissue containing a first plurality of cell types having a first proportionality of numbers, at least one second functionality operative on at least some of the first plurality of cell types, adaptive control functionality operative to control the operation of the at least one second functionality as a function of at least some of the numbers, and long-term monitored culturing functionality for storing the at least some of the first plurality of cell types.

There is still further provided in accordance with a still further embodiment of the present invention an automated cell bank generating system including a first functionality which receives at least one tissue containing a first plurality of cell types having a first proportionality of numbers, at least one second functionality operative on a second plurality of cell types being at least some of the first plurality of cell types and having a second proportionality of numbers which varies over time, monitoring functionality operative to monitor variations in the numbers of the second plurality of cell types, adaptive control functionality operative to control the operation of the at least one second functionality as a function of at least some of the variations in the numbers of the second plurality of cell types and storage functionality for storing the second plurality of cell types.

There is also provided in accordance with another embodiment of the present invention an automated cell bank generating system including a first functionality which receives at least one tissue containing a first plurality of cell types having a first proportionality of numbers, at least one second functionality operative on at least some of the first plurality of cell types, monitoring functionality operative to monitor differentiation of the at least some of the first plurality of cell types, adaptive control functionality operative to control the operation of the at least one second functionality in response to at least one output of the monitoring functionality and storage functionality for storing the at least some of the first plurality of cell types.

There is further provided in accordance with a further embodiment of the present invention an automated cell bank generating system including a first functionality which receives at least one tissue containing a first plurality of cell types having a first proportionality of numbers, at least one second functionality operative with respect to at least some of the first plurality of cell types to at least one of add, mix or remove at least one material, monitoring functionality operative to monitor the at least some of the first plurality of cell types, adaptive control functionality operative to control the operation of the at least one second functionality in response to at least one output of the monitoring functionality and storage functionality for storing the at least some of the first plurality of cell types.

There is still further provided in accordance with a still further embodiment of the present invention an automated autologous cell therapy system including functionality which receives at least one tissue containing a multiplicity of cells belonging to multiple cell types, automatic functionality which modulates (e.g., increases or decreases) both the proportion of cells of at least one of the multiple cell types as compared with at least another of the multiple cell types and also modulates (e.g., increases or decreases) the absolute number of cells of the at least one of the multiple cell types and autologous implantation functionality for supplying the at least one of the multiple cell types to a source of the at least one tissue. The implantation functionality may include, for example, a syringe, a tube, a catheter, or a therapeutic administration device.

There is additionally provided in accordance with an additional embodiment of the present invention an automated autologous cell therapy system including functionality which receives at least one tissue containing a multiplicity of cells belonging to multiple cell types, automatic functionality including cell culturing which modulates (e.g., increases or decreases) the proportion of cells of at least one of the multiple cell types as compared with at least another of the multiple cell types and autologous implantation functionality for supplying the at least one of the multiple cell types to a source of the at least one tissue.

There is further provided in accordance with a further embodiment of the present invention an automated autologous cell therapy system including a first functionality which receives at least one tissue containing a first plurality of cell types having a first proportionality of numbers, at least one second functionality operative on at least some of the first plurality of cell types, adaptive control functionality operative to control the operation of the at least one second functionality as a function of at least some of the numbers and autologous implantation functionality for supplying the at least some of the first plurality of cell types to a source of the at least one tissue.

There is also provided in accordance with another embodiment of the present invention an automated autologous cell therapy system including a first functionality which receives at least one tissue containing a first plurality of cell types having a first proportionality of numbers, at least one second functionality operative on a second plurality of cell types being at least some of the first plurality of cell types and having a second proportionality of numbers which varies over time, monitoring functionality operative to monitor variations in the numbers of the second plurality of cell types, adaptive control functionality operative to control the operation of the at least one second functionality as a function of at least some of the variations in the numbers of the second plurality of cell types and autologous implantation functionality for supplying the second plurality of cell types to a source of the at least one tissue.

There is additionally provided in accordance with an additional embodiment of the present invention an automated autologous cell therapy system including a first functionality which receives at least one tissue containing a first plurality of cell types having a first proportionality of numbers, at least one second functionality operative on at least some of the first plurality of cell types, monitoring functionality operative to monitor differentiation of the at least some of the first plurality of cell types, adaptive control functionality operative to control the operation of the at least one second functionality in response to at least one output of the monitoring functionality and autologous implantation functionality for supplying the at least some of the first plurality of cell types to a source of the at least one tissue.

There is further provided in accordance with a further embodiment of the present invention an automated autologous cell therapy system including a first functionality which receives at least one tissue containing a first plurality of cell types having a first proportionality of numbers, at least one second functionality operative with respect to at least some of the first plurality of cell types to at least one of add, mix or remove at least one material, monitoring functionality operative to monitor the at least some of the first plurality of cell types, adaptive control functionality operative to control the operation of the at least one second functionality in response to at least one output of the monitoring functionality and autologous implantation functionality for supplying the at least some of the first plurality of cell types to a source of the at least one tissue.

There is still further provided in accordance with a still further embodiment of the present invention an automated non-autologous cell therapy system including functionality which receives at least one tissue containing a multiplicity of cells belonging to multiple cell types, automatic functionality which modulates (e.g., increases or decreases) both the proportion of cells of at least one of the multiple cell types as compared with at least another of the multiple cell types and also modulates (e.g., increases or decreases) the absolute number of cells of the at least one of the multiple cell types and non-autologous implantation functionality for supplying the at least one of the multiple cell types to a recipient other than a source of the at least one tissue.

There is yet further provided in accordance with yet a further embodiment of the present invention an automated non-autologous cell therapy system including functionality which receives at least one tissue containing a multiplicity of cells belonging to multiple cell types, automatic functionality including cell culturing which modulates (e.g., increases or decreases) the proportion of cells of at least one of the multiple cell types as compared with at least another of the multiple cell types and non-autologous implantation functionality for supplying the at least one of the multiple cell types to a recipient other than a source of the at least one tissue.

There is also provided in accordance with another embodiment of the present invention an automated non-autologous cell therapy system including a first functionality which receives at least one tissue containing a first plurality of cell types having a first proportionality of numbers, at least one second functionality operative on at least some of the first plurality of cell types, adaptive control functionality operative to control the operation of the at least one second functionality as a function of at least some of the numbers and non-autologous implantation functionality for supplying the at least some of the first plurality of cell types to a recipient other than a source of the at least one tissue.

There is additionally provided in accordance with an additionally embodiment of the present invention an automated non-autologous cell therapy system including a first functionality which receives at least one tissue containing a first plurality of cell types having a first proportionality of numbers, at least one second functionality operative on a second plurality of cell types being at least some of the first plurality of cell types and having a second proportionality of numbers which varies over time, monitoring functionality operative to monitor variations in the numbers of the second plurality of cell types, adaptive control functionality operative to control the operation of the at least one second functionality as a function of at least some of the variations in the numbers of the second plurality of cell types and non-autologous implantation functionality for supplying the second plurality of cell types to a recipient other than a source of the at least one tissue.

There is further provided in accordance with a further embodiment of the present invention an automated non-autologous cell therapy system including a first functionality which receives at least one tissue containing a first plurality of cell types having a first proportionality of numbers, at least one second functionality operative on at least some of the first plurality of cell types, monitoring functionality operative to monitor differentiation of the at least some of the first plurality of cell types, adaptive control functionality operative to control the operation of the at least one second functionality in response to at least one output of the monitoring functionality and non-autologous implantation functionality for supplying the at least some of the first plurality of cell types to a recipient other than a source of the at least one tissue.

There is still further provided in accordance with a still further embodiment of the present invention an automated non-autologous cell therapy system including a first functionality which receives at least one tissue containing a first plurality of cell types having a first proportionality of numbers, at least one second functionality operative with respect to at least some of the first plurality of cell types to at least one of add, mix or remove at least one material, monitoring functionality operative to monitor the at least some of the first plurality of cell types, adaptive control functionality operative to control the operation of the at least one second functionality in response to at least one output of the monitoring functionality and non-autologous implantation functionality for supplying the at least some of the first plurality of cell types to a recipient other than a source of the at least one tissue.

In accordance with another embodiment of the present invention, the automated autologous cell therapy system is operative to supply the at least some of the first plurality or at least some of the second plurality of cell types in the form of drops, cream, spray or freeze-dried products.

In accordance with another embodiment of the present invention, the automated non-autologous cell therapy system is operative to formulate the at least some of the first plurality or at least some of the second plurality of cell types in the form of drops, cream, spray or freeze-dried products.

In accordance with yet another embodiment of the present invention the autologous implantation functionality for supplying the at least some of the first plurality or at least some of the second plurality of cell types to a recipient other than a source of the at least one tissue includes automated packaging functionality.

In accordance with yet another embodiment of the present invention the system includes automated packaging functionality.

In accordance with an embodiment of the present invention the automated system also includes quality control functionality. Typically, the quality control functionality interfaces with the monitoring functionality and the adaptive control functionality. Additionally or alternatively, the monitoring functionality includes optical inspection functionality.

In accordance with another embodiment of the present invention, the monitoring functionality is operative to remotely monitor the cell types and/or other parameters. Such parameters may include, for example, the status of the system's functional sub-units (e.g., based on self-testing of incubator CO2 levels or proper centrifuge motor operation), expiration dates of various components or materials (e.g., VEGF), or calibration of hardware components (e.g., pipettes). Alternatively, the monitoring functionality is operative to monitor the cell types and the other parameters on-site.

In accordance with another embodiment of the present invention the quality control functionality employs sampling functionality for providing quality control samples and functionality for evaluating the quality control samples. Typically, the functionality for evaluating the quality control samples includes at least one of sterility analysis, flow cytometry analysis, immunoassays and at least one tissue culture based tests.

In accordance with yet another embodiment of the present invention the adaptive control functionality is operative to perform at least one of the following steps in response to inputs received from the monitoring functionality: vary process parameters; vary process flow; verify process parameters; order that certain batches of cells or other materials be discarded; and provide warnings to supervisory personnel and displays of system and methodology parameters.

In accordance with yet another embodiment of the present invention the adaptive control functionality is operative to perform at least one of the following steps in response to inputs received from the functionality for evaluating the quality control samples: vary process parameters; vary process flow; verify process parameters; order that certain batches of cells or other materials be discarded; and provide warnings to supervisory personnel and displays of system and methodology parameters.

In accordance with still another embodiment of the present invention the adaptive control functionality interfaces with at least one of administration software and at least one historical database. Additionally or alternatively, the adaptive control functionality is operative to perform at least one of the steps in real-time. Typically, the adaptive control functionality is operative to perform at least one of the steps in response to inputs received from the at least one historical database. As a further alternative, the adaptive control functionality is operative to perform at least one of the steps in response to inputs received from the administration software.

In accordance with a further embodiment of the present invention the at least one of the multiple cell types is utilized in a cellular therapeutic product. Alternatively or additionally, the at least one of the multiple cell types is utilized in a diagnostic tool. As a further alternative, the at least one of the multiple cell types is utilized in a biological research tool. Alternatively or additionally, the at least one of the multiple cell types is utilized in a biological development tool.

There is also provided in accordance with an embodiment of the present invention a self-scraping cell culture assembly including a generally annular dish defining a generally flat, circularly-shaped cell growth surface, a cover arranged for sealing engagement with the annular dish and at least one scraper blade mechanically associated with the cover, whereby rotation of the cover relative to the dish provides scraping of cells from the circularly-shaped cell growth surface.

In accordance with an embodiment of the present invention the cover is formed with at least one of a septum element operative for insertion of a pipetting device into the annular dish, and a vent.

There is also provided in accordance with an embodiment of the present invention a method for automated cell processing including receiving at least one tissue containing a multiplicity of cells belonging to multiple cell types and automatically increasing or otherwise modulating both the proportion of cells of at least one of the multiple cell types as compared with at least another of the multiple cell types and the absolute number of cells of the at least one of the multiple cell types.

There is additionally provided in accordance with an additional embodiment of the present invention a method for automated cell processing including receiving at least one tissue containing a multiplicity of cells belonging to multiple cell types and automatically culturing the cells thereby increasing or otherwise modulating the proportion of cells of at least one of the multiple cell types as compared with at least another of the multiple cell types.

There is further provided in accordance with a further embodiment of the present invention a method for automated cell processing including receiving at least one tissue containing a first plurality of cell types having a first proportionality of numbers, operating on at least some of the first plurality of cell types and controlling the operating as a function of at least some of the numbers.

There is still further provided in accordance with a still further embodiment of the present invention a method for automated cell processing including receiving at least one tissue containing a first plurality of cell types having a first proportionality of numbers, operating on a second plurality of cell types being at least some of the first plurality of cell types and having a second proportionality of numbers which varies over time, monitoring variations in the numbers of the second plurality of cell types, and controlling the operating as a function of at least some of the variations in the numbers of the second plurality of cell types.

There is also provided in accordance with another embodiment of the present invention a method for automated cell processing including receiving at least one tissue containing a first plurality of cell types having a first proportionality of numbers, operating on at least some of the first plurality of cell types, monitoring differentiation of the at least some of the first plurality of cell types and controlling the operating in response to at least one output of the monitoring functionality.

There is additionally provided in accordance with an additionally embodiment of the present invention a method for automated cell processing including receiving at least one tissue containing a first plurality of cell types having a first proportionality of numbers, at least one of adding at least one material (e.g., cells, liquids, molecules) to at least some of the first plurality of cell types and removing at least one material from at least some of the first plurality of cell types, monitoring the at least some of the first plurality of cell types and controlling the operating in response to at least one output of the monitoring functionality.

There is further provided in accordance with a further embodiment of the present invention a method for automated generation of a cell bank including receiving at least one tissue containing a multiplicity of cells belonging to multiple cell types, automatically increasing or otherwise modulating both the proportion of cells of at least one of the multiple cell types as compared with at least another of the multiple cell types and the absolute number of cells of the at least one of the multiple cell types and storing the at least one of the multiple cell types.

There is still further provided in accordance with a still further embodiment of the present invention a method for automated generation of a cell bank including receiving at least one tissue containing a multiplicity of cells belonging to multiple cell types, automatically culturing the cells thereby increasing or otherwise modulating the proportion of cells of at least one of the multiple cell types as compared with at least another of the multiple cell types and storing the at least one of the multiple cell types.

There is also provided in accordance with another embodiment of the present invention a method for automated generation of a cell bank including receiving at least one tissue containing a first plurality of cell types having a first proportionality of numbers, operating on at least some of the first plurality of cell types, controlling the operating as a function of at least some of the numbers and storing the at least one of the multiple cell types.

There is additionally provided in accordance with an additional embodiment of the present invention a method for automated generation of a cell bank including receiving at least one tissue containing a first plurality of cell types having a first proportionality of numbers, operating on a second plurality of cell types being at least some of the first plurality of cell types and having a second proportionality of numbers which varies over time, monitoring variations in the numbers of the second plurality of cell types, controlling the operating as a function of at least some of the variations in the numbers of the second plurality of cell types and storing the at least one of the multiple cell types.

There is further provided in accordance with a further embodiment of the present invention a method for automated generation of a cell bank including receiving at least one tissue containing a first plurality of cell types having a first proportionality of numbers, operating on at least some of the first plurality of cell types, monitoring differentiation of the at least some of the first plurality of cell types, controlling the operating in response to at least one output of the monitoring functionality and storing the at least one of the multiple cell types.

There is still further provided in accordance with a still further embodiment of the present invention a method for automated generation of a cell bank including receiving at least one tissue containing a first plurality of cell types having a first proportionality of numbers, at least one of adding at least one material to at least some of the first plurality of cell types and removing at least one material from at least some of the first plurality of cell types, monitoring the at least some of the first plurality of cell types, controlling the operating in response to at least one output of the monitoring functionality and storing the at least one of the multiple cell types.

There is also provided in accordance with another embodiment of the present invention a method for automated autologous cell therapy including receiving at least one tissue containing a multiplicity of cells belonging to multiple cell types, automatically increasing or otherwise modulating both the proportion of cells of at least one of the multiple cell types as compared with at least another of the multiple cell types and the absolute number of cells of the at least one of the multiple cell types and supplying the at least one of the multiple cell types to a source of the at least one tissue.

There is additionally provided in accordance with an additional embodiment of the present invention a method for automated autologous cell therapy including receiving at least one tissue containing a multiplicity of cells belonging to multiple cell types, automatically culturing the cells thereby increasing or otherwise modulating the proportion of cells of at least one of the multiple cell types as compared with at least another of the multiple cell types and supplying the at least one of the multiple cell types to a source of the at least one tissue.

There is further provided in accordance with a further embodiment of the present invention a method for automated autologous cell therapy including receiving at least one tissue containing a first plurality of cell types having a first proportionality of numbers, operating on at least some of the first plurality of cell types, controlling the operating as a function of at least some of the numbers and supplying the at least one of the multiple cell types to a source of the at least one tissue.

There is still further provided in accordance with a still further embodiment of the present invention a method for automated autologous cell therapy including receiving at least one tissue containing a first plurality of cell types having a first proportionality of numbers, operating on a second plurality of cell types being at least some of the first plurality of cell types and having a second proportionality of numbers which varies over time, monitoring variations in the numbers of the second plurality of cell types, controlling the operating as a function of at least some of the variations in the numbers of the second plurality of cell types and supplying the at least one of the multiple cell types to a source of the at least one tissue.

There is also provided in accordance with another embodiment of the present invention a method for automated autologous cell therapy including receiving at least one tissue containing a first plurality of cell types having a first proportionality of numbers, operating on at least some of the first plurality of cell types, monitoring differentiation of the at least some of the first plurality of cell types, controlling the operating in response to at least one output of the monitoring functionality and supplying the at least one of the multiple cell types to a source of the at least one tissue.

There is additionally provided in accordance with an additional embodiment of the present invention a method for automated autologous cell therapy including receiving at least one tissue containing a first plurality of cell types having a first proportionality of numbers, at least one of adding at least one material to at least some of the first plurality of cell types and removing at least one material from at least some of the first plurality of cell types, monitoring the at least some of the first plurality of cell types, controlling the operating in response to at least one output of the monitoring functionality and supplying the at least one of the multiple cell types to a source of the at least one tissue.

There is further provided in accordance with a further embodiment of the present invention a method for automated non-autologous cell therapy including receiving at least one tissue containing a multiplicity of cells belonging to multiple cell types, automatically increasing or otherwise modulating both the proportion of cells of at least one of the multiple cell types as compared with at least another of the multiple cell types and the absolute number of cells of the at least one of the multiple cell types and supplying the at least one of the multiple cell types to a recipient other than a source of the at least one tissue.

There is still further provided in accordance with a still further embodiment of the present invention a method for automated non-autologous cell therapy including receiving at least one tissue containing a multiplicity of cells belonging to multiple cell types, automatically culturing the cells thereby increasing or otherwise modulating the proportion of cells of at least one of the multiple cell types as compared with at least another of the multiple cell types and supplying the at least one of the multiple cell types to a recipient other than a source of the at least one tissue.

There is also provided in accordance with another embodiment of the present invention a method for automated non-autologous cell therapy including receiving at least one tissue containing a first plurality of cell types having a first proportionality of numbers, operating on at least some of the first plurality of cell types, controlling the operating as a function of at least some of the numbers and supplying the at least one of the multiple cell types to a recipient other than a source of the at least one tissue.

There is additionally provided in accordance with an additional embodiment of the present invention a method for automated non-autologous cell therapy including receiving at least one tissue containing a first plurality of cell types having a first proportionality of numbers, operating on a second plurality of cell types being at least some of the first plurality of cell types and having a second proportionality of numbers which varies over time, monitoring variations in the numbers of the second plurality of cell types, controlling the operating as a function of at least some of the variations in the numbers of the second plurality of cell types and supplying the at least one of the multiple cell types to a recipient other than a source of the at least one tissue.

There is further provided in accordance with a further embodiment of the present invention a method for automated non-autologous cell therapy including receiving at least one tissue containing a first plurality of cell types having a first proportionality of numbers, operating on at least some of the first plurality of cell types, monitoring differentiation of the at least some of the first plurality of cell types, controlling the operating in response to at least one output of the monitoring functionality and supplying the at least one of the multiple cell types to a recipient other than a source of the at least one tissue.

There is still further provided in accordance with a still further embodiment of the present invention a method for automated non-autologous cell therapy including receiving at least one tissue containing a first plurality of cell types having a first proportionality of numbers, at least one of adding at least one material to at least some of the first plurality of cell types and removing at least one material from at least some of the first plurality of cell types, monitoring the at least some of the first plurality of cell types, controlling the operating in response to at least one output of the monitoring functionality and supplying the at least one of the multiple cell types to a recipient other than a source of the at least one tissue.

There is also provided, in accordance with an embodiment of the invention, an automated cell processing system including:
functionality which receives at least one tissue containing a multiplicity of cells belonging to multiple cell types; and
automatic functionality which modulates both the proportion of cells of at least one of said multiple cell types as compared with at least another of said multiple cell types and also modulates the absolute number of cells of said at least one of said multiple cell types.

There is additionally provided, in accordance with an embodiment of the invention, an automated cell processing system including:
functionality which receives at least one tissue containing a multiplicity of cells belonging to multiple cell types; and
automatic functionality including cell culturing which modulates the proportion of cells of at least one of said multiple cell types as compared with at least another of said multiple cell types.

In an embodiment, the system is operative to formulate at least some of said cells in a form selected from the group consisting of: drops, cream, spray and freeze-dried.

In an embodiment, the automatic functionality including packaging functionality for packaging at least one of said multiple cell types.

There is yet additionally provided, in accordance with an embodiment of the invention, an automated cell bank generating system including:
functionality which receives at least one tissue containing a multiplicity of cells belonging to multiple cell types;
automatic functionality which modulates both the proportion of cells of at least one of said multiple cell types as compared with at least another of said multiple cell types and also modulates the absolute number of cells of said at least one of said multiple cell types; and
storage functionality for storing said at least one of said multiple cell types.

There is still additionally provided, in accordance with an embodiment of the invention, an automated cell bank generating system including:
functionality which receives at least one tissue containing a multiplicity of cells belonging to multiple cell types;
automatic functionality including cell culturing which modulates the proportion of cells of at least one of said multiple cell types as compared with at least another of said multiple cell types; and
storage functionality for storing said at least one of said multiple cell types.

There is also provided, in accordance with an embodiment of the invention, an automated autologous cell therapy system including:
functionality which receives at least one tissue containing a multiplicity of cells belonging to multiple cell types;
automatic functionality which modulates both the proportion of cells of at least one of said multiple cell types as compared with at least another of said multiple cell types and also modulates the absolute number of cells of said at least one of said multiple cell types; and
autologous implantation functionality for supplying said at least one of said multiple cell types to a source of said at least one tissue.

There is further provided, in accordance with an embodiment of the invention, an automated autologous cell therapy system including:
functionality which receives at least one tissue containing a multiplicity of cells belonging to multiple cell types;
automatic functionality including cell culturing which modulates the proportion of cells of at least one of said multiple cell types as compared with at least another of said multiple cell types; and
autologous implantation functionality for supplying said at least one of said multiple cell types to a source of said at least one tissue.

There is yet further provided, in accordance with an embodiment of the invention, an automated non-autologous cell therapy system including:
functionality which receives at least one tissue containing a multiplicity of cells belonging to multiple cell types;
automatic functionality which modulates both the proportion of cells of at least one of said multiple cell types as compared with at least another of said multiple cell types and also modulates the absolute number of cells of said at least one of said multiple cell types; and
non-autologous implantation functionality for supplying said at least one of said multiple cell types to a recipient other than a source of said at least one tissue.

There is still further provided, in accordance with an embodiment of the invention, an automated non-autologous cell therapy system including:
functionality which receives at least one tissue containing a multiplicity of cells belonging to multiple cell types;
automatic functionality including cell culturing which modulates the proportion of cells of at least one of said multiple cell types as compared with at least another of said multiple cell types; and
non-autologous implantation functionality for supplying said at least one of said multiple cell types to a recipient other than a source of said at least one tissue.

In an embodiment, the system includes automated packaging functionality.

In an embodiment, said automatic functionality:

increases said proportion of cells;

increases said absolute number of cells; or

increases said proportion of cells and said absolute number of cells.

In an embodiment, the system also includes at least one of monitoring functionality and adaptive control functionality.

In an embodiment, the system also includes quality control functionality.

In an embodiment, said quality control functionality interfaces with said monitoring functionality and said adaptive control functionality.

In an embodiment, said quality control functionality employs sampling functionality for providing quality control samples and functionality for evaluating said quality control samples. In an embodiment, said functionality for evaluating said quality control samples includes at least one of sterility analysis, gram stain analysis, endotoxin analysis, mycoplasma analysis, tube formation assays, flow cytometry analysis, immunoassays and tissue culture based tests.

There is also provided, in accordance with an embodiment of the invention, an automated cell processing system including:
a first functionality which receives a culture including at least one tissue containing a first plurality of cell types;
at least one second functionality operative on at least some of said first plurality of cell types; and
adaptive control functionality operative to control the operation of said at least one second functionality as a function of at least one parameter of said culture.

There is additionally provided, in accordance with an embodiment of the invention, an automated cell processing system including:
a first functionality which receives a culture including at least one tissue containing a first plurality of cell types;
at least one second functionality, operative on a second plurality of cell types being at least some of said first plurality of cell types, and having at least one parameter associated therewith that varies over time;
monitoring functionality operative to monitor variations in said at least one parameter; and
adaptive control functionality operative to control the operation of said at least one second functionality as a function of at least some of said variations in said at least one parameter.

In an embodiment, the parameter includes one, some, or all of the following:

a level of response of said cells to an external input;

a characteristic of a secretion of said cells;

protein expression within said cells;

a genetic composition of said cells;

a level of viability of said cells;

a level of mortality of said cells;

a level of necrosis of said cells;

a level of apoptosis of said cells;

a level of proliferation of said cells; and

morphology of said cells.

### BRIEF DESCRIPTION OF THE DRAWINGS

The present invention will be understood and appreciated more fully from the following detailed description, taken in conjunction with the drawings in which:

FIG. 1 is a simplified schematic illustration of an automated cell culturing system and methodology constructed and operative in accordance with an embodiment of the present invention;

FIG. 2 is a simplified flow chart of various steps in the methodology illustrated generally in FIG. 1;

FIGS. 3A and 3B are detailed flow charts illustrating steps in the methodology illustrated generally in FIGS. 1 & 2; and

FIGS. 4A and 4B are respective simplified pictorial and sectional illustrations of a self-scraping cell culture assembly constructed and operative in accordance with an embodiment of the present invention.

### DETAILED DESCRIPTION OF EMBODIMENTS

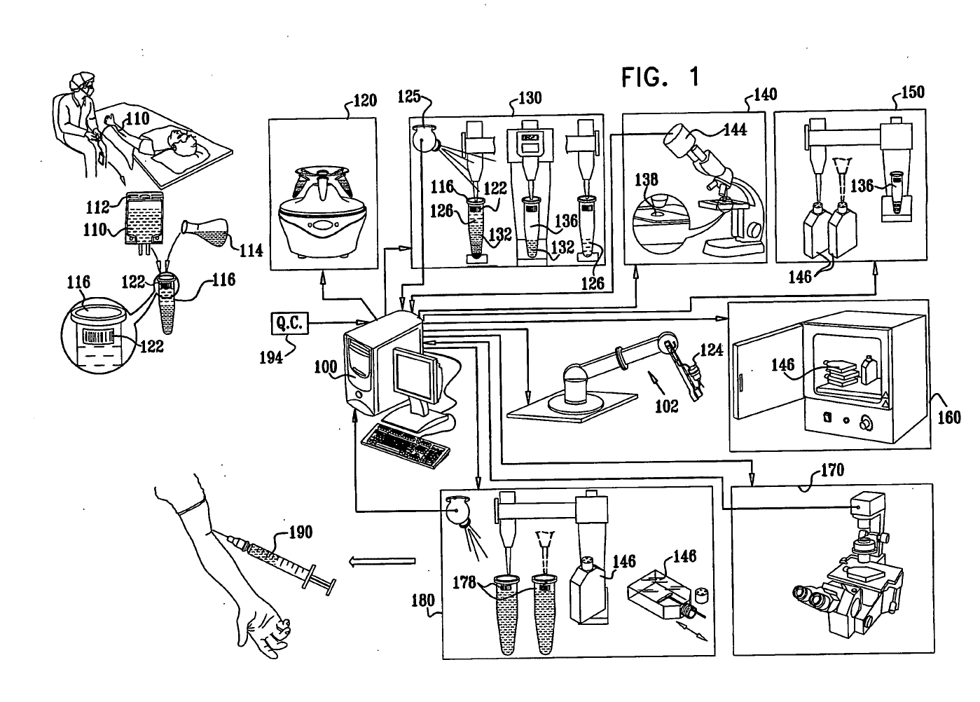

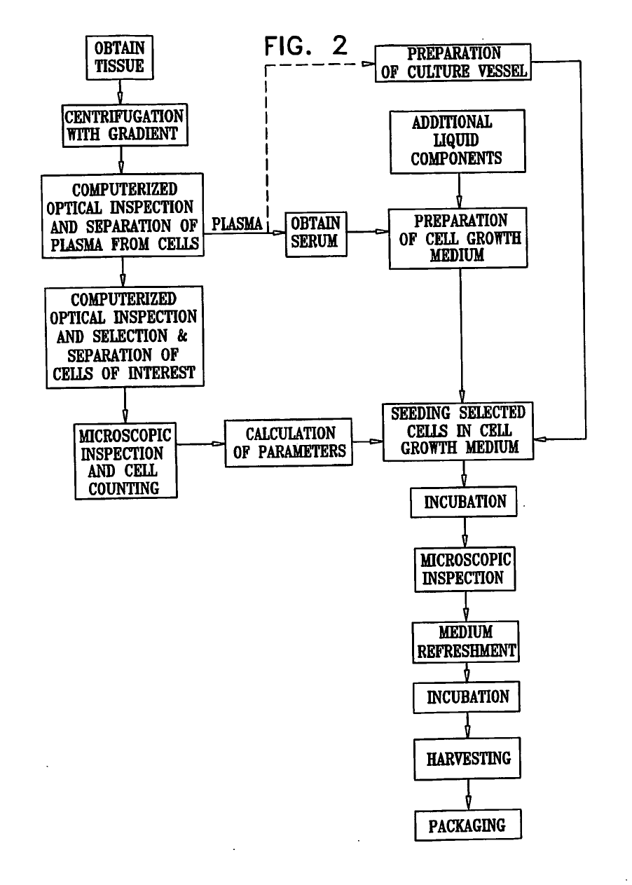

Reference is now made to FIG. 1, which is a simplified schematic illustration of an automated cell culturing system and methodology constructed and operative in accordance with an embodiment of the present invention. As seen generally in FIG. 1, the automated cell culturing system includes an interactive computer controller 100 which monitors and controls various automated cell culturing operations based, inter alia, on visual feedback. The automated cell culturing operations employ a plurality of operational modules, which will now be described, as well as mechanized transfer functionality, typically embodied in a computer-controlled robotic subsystem, schematically illustrated in FIG. 1 and designated by reference numeral 102. The general methodology of embodiments of the present invention is illustrated in FIG. 2.

It is a particular feature of some embodiments of the present invention that the automated cell culturing system and methodology includes functionality which receives at least one tissue containing a multiplicity of cells belonging to multiple cell types and an automatic functionality which modulates (e.g., increases or decreases) both the proportion of cells of at least one of the multiple cell types as compared with at least another of the multiple cell types and also modulates (e.g., increases or decreases) the absolute number of cells of the at least one of the multiple cell types.

The tissue or tissues that are used as a starting material in the system and methodology of some embodiments of the present invention may be any suitable type of tissue, such as an animal or plant tissue. The animal tissue may be human tissue or tissue of another mammal or of any other suitable animal. The cell types which are produced by the system and methodology of some embodiments of the present invention may be introduced into tissue of any suitable organism, such as, for example, humans or other mammals. The cell types may be reintroduced into the same organism from which the tissue or tissues were removed, which is known as autologous implantation, or alternatively introduced to another organism of the same genus, such as human to human, which is known as allogeneic implantation, or introduced to another organism not of the same genus, which is known as xenogeneic implantation, both of the latter being classifiable as non-autologous implantation.

It is also a particular feature of some embodiments of the present invention that the automated cell culturing system and methodology includes functionality which receives at least one tissue containing a multiplicity of cells belonging to multiple cell types and automatic functionality including cell culturing which modulates (e.g., increases or decreases) the proportion of cells of at least one of the multiple cell types as compared with at least another of the multiple cell types.

It is additionally a particular feature of some embodiments of some embodiments of the present invention that the automated cell culturing system and methodology includes a first functionality which receives at least one tissue containing a first plurality of cell types having a first proportionality of numbers, at least one second functionality operative on at least some of the first plurality of cell types and adaptive control functionality operative to control the operation of the at least one second functionality as a function of at least some of the numbers defining the first proportionality.

It is further a particular feature of some embodiments of the present invention that the automated cell culturing system and methodology includes a first functionality which receives at least one tissue containing a first plurality of cell types having a first proportionality of numbers, at least one second functionality operative on another plurality of cell types being at least some of the first plurality of cell types and having a second proportionality of numbers which varies over time, monitoring functionality operative to monitor variations in the numbers of the second plurality of cell types and adaptive control functionality operative to control the operation of the at least one second functionality as a function of at least some of the variations in the numbers of the second plurality of cell types.

It is also a particular feature of some embodiments of the present invention that the automated cell culturing system and methodology includes a first functionality which receives at least one tissue containing a first plurality of cell types having a first proportionality of numbers, at least one second functionality operative on at least some of the first plurality of cell types, monitoring functionality operative to monitor differentiation of the at least some of the first plurality of cell types and adaptive control functionality operative to control the operation of the at least one second functionality in response to at least one output of the monitoring functionality.

It is additionally a particular feature of some embodiments of the present invention that the automated cell culturing system and methodology includes a first functionality which receives at least one tissue containing a first plurality of cell types having a first proportionality of numbers, at least one second functionality operative with respect to at least some of the first plurality of cell types to at least one of add, mix or remove at least one material, monitoring functionality operative to monitor the at least some of the first plurality of cell types and adaptive control functionality operative to control the operation of said at least one second functionality in response to at least one output of the monitoring functionality.

It is a further particular feature of some embodiments of the present invention that the automated cell culturing system and methodology additionally includes at least one of storage functionality for storing at least one of the multiple cell types, autologous implantation functionality for supplying at least one of the multiple cell types to a source of the at least one tissue and non-autologous implantation functionality for supplying at least some of the first plurality of cell types to a recipient other than a source of the at least one tissue.

Referring additionally to the general methodology as it appears in FIG. 2, in accordance with an embodiment of the present invention, one or more tissues are obtained from any suitable source, such as the blood of a patient, as indicated schematically in FIG. 1 at reference numeral 110, and is stored in a conventional blood storage bag 112, typically containing an anticoagulant such as sodium citrate. Alternatively, tissue may be obtained from any other suitable source, such as blood from another organism, bone marrow from the patient or from another organism, umbilical cord blood, stem cells, embryonic material, fetal material and other organ derived tissue.

In the illustrated embodiment, where a patient's blood is employed as a source of tissue, typically, blood from the storage bag 112 is laid on a polysaccharide gradient 114 which comprises a liquid which causes the separation of cells according to their sedimentation rate, based on their density. The combination, typically located in one or more tubes 116, may then be centrifuged in a centrifuge module 120. Following centrifugation, different layers which are formed in tubes 116 are optically differentiable by the relative location of the layer and the grey level or color gradient of light reflected therefrom.

Typically, each vessel, which contains tissue or derivatives thereof, is individually and machine-readably identified by an individual identification code, such as a bar code 122. Typically, all of the vessels containing tissue or derivatives thereof from a given source, such as an individual patient are coded to indicate their common provenance. The individual identification codes, such as bar codes 122, are typically machine read by bar code readers, such as a bar code reader 124 which may be associated with computer-controlled robotic subsystem 102. Alternatively or additionally, the bar codes 122 may be read by optical inspection devices which may be incorporated within the system at appropriate locations. Outputs of bar code reading are supplied to interactive computer controller 100.

Downstream of the foregoing centrifugation step, the contents of one or more tubes 116 are typically identified by optical inspection functionality 125, typically embodied in a conventional CCD camera. Plasma 126 is typically separated from the remainder of the contents of one or more tubes 116, in a pipetting module 130, according to instructions received from interactive computer controller 100 based on outputs of the optical inspection of functionality 125 which is included in the pipetting module 130. In practice, a plurality of pipetting modules 130, typically each including an optical inspection functionality 125, are included in the system and may be designed to handle different volumes, speeds and types of liquids and to provide different functions, such as aspiration, addition of liquid, dilution and suspension. The term “pipetting” is here employed in a broader sense than usual to include but not to be limited to any suitable type of precise dispensing useful in the present system and methodology. As will be described hereinbelow, the plasma may be used to produce serum useful in the system and methodology of some embodiments of the present invention.

The pipetting module 130 is typically also employed downstream of the removal of plasma 126 from one or more tubes 116, for removing a desired layer of cells 132, which are typically differentiable by the relative location of the layer and the grey level or color gradient of light reflected therefrom. In the illustrated embodiment of the invention, layer 132 comprises peripheral blood mononuclear cells (PBMC).

Optical inspection functionality 125 is capable, inter alia, of viewing the contents of one or more tubes 116 at various stages of separation of the contents thereof and to provide output, enabling, inter alia, determination of the aforesaid relative location and grey level. Alternatively or additionally, a color camera may be employed together with a polychromatic gradient.

The cells in layer 132 are alternatively or additionally pipetted onto an additional gradient for further cell enrichment. The resulting cell layer or layers are viewed by optical inspection functionality 125, which determines the relative location and grey level of the aforesaid layer or layers. For some applications, the location of the layer(s) is identified by comparison with another tube containing cellular density marker beads or another type of marker. Cells in layer(s) designated for removal are identified based on the locations of the marker beads in the other tube having corresponding densities.

The cells in layer 132, or the cells resulting from any further enrichment, are uniformly suspended in a known volume of a suitable liquid placed in a tube 136, typically by a pipetting module 130. The suspended cells may be subjected to further sedimentation and purification process, following which optical inspection functionalities, such as optical inspection functionality 125, view the sedimented cells and provide an output to interactive computer controller 100, typically in order to, inter alia, determine a volume of supernatant to be collected or discarded. The cells are then resuspended, typically in a known volume of liquid. A known volume sample 138 of the resuspended cells, typically in tube 136, is then examined in a microscopic inspection module 140. Microscopic inspection module 140 typically includes optical inspection functionality 144, typically embodied in a conventional CCD camera, which is capable, inter alia, of viewing a magnified sample, inter alia to determine the numbers and viability of the cells in the sample 138. Outputs of optical inspection functionality 144 are supplied to interactive computer controller 100.

Based on the outputs of optical inspection functionality 144, interactive computer controller 100 calculates a number of culture vessels, an initial cell concentration and a suitable quantity of cell growth medium and ingredients that will be required.

At this stage or prior thereto a required quantity of culture vessels 146 is prepared, typically by coating an interior planar surface thereof with a cell adhesion enhancer, typically including proteins derived from the plasma 126 separated from the contents of one or more tubes 116. Alternatively, the coating may be any other suitable cell adhesion enhancer, such as plasma other than from the patient or a material manufactured from proteins derived therefrom, for example Fibronectin. An alternative cell adhesion enhancer may include an antibody, a growth enhancing molecule such as collagen and other growth enhancing molecules. Coating of culture vessels with Fibronectin and other suitable factors is described in U.S. Provisional Patent Application No. 60/576,266 which is incorporated herein by reference.

A suitable cell growth medium is typically prepared on a just-in-time basis in a pipetting module similar to pipetting module 130. A cell growth medium may be prepared using serum derived from the plasma 126 of the patient. Preparation of serum from plasma is described in U.S. Provisional Patent Application No. 60/576,266 which is incorporated herein by reference.

Alternatively, the serum can be manufactured from plasma other than from the patient, or purchased from commercial sources.

The cell growth medium is prepared using the serum and a plurality of additional liquid and/or soluble components.

A portion of the cell growth medium is supplied to each of the coated culture vessels 146. Another portion of the cell growth medium is employed to suspend the cells in tube 136 and the suspended cells are dispensed into the various culture vessels 146, typically by a suitable pipetting module 150. The remaining cell growth medium is suitably refrigerated.

The cells in the culture vessels 146 are then incubated, in an incubator module 160, typically at 37° C. and in a humidified environment including 5% CO₂, typically for three days. Typically the culture vessel 146 is subjected to periodic microscopic inspection at a vessel microscopic inspection module 170, typically including an inverted microscope system, to examine cell culture status, which supplies an output to interactive computer controller 100.

The cell growth medium, previously stored under refrigeration, is used for refreshing the cell growth medium in the culture vessels 146, typically every 2-4 days.

Following suitable incubation, the cells are harvested from the interior of the culture vessels 146, as by chemical or mechanical detachment, such as scraping, or use of trypsin, and are suspended in one or more tubes 178 which are filled with a known volume of liquid by a pipetting module 180 which is similar to pipetting module 130. The scraped culture vessels 146 are typically subjected to microscopic inspection at vessel microscopic inspection module 170, inter alia to ensure that all the desired cells have been collected from the vessels 146.

The harvested cells are counted, typically using the microscopic inspection module 140, inter alia to determine the numbers and viability of the cells harvested from the culture vessels 146. Outputs of optical inspection functionality 144 are supplied to interactive computer controller 100.

At this stage, based on the outputs of optical inspection functionality 144, the interactive computer controller 100 typically calculates a suitable number of delivery containers, such as pre-fillable syringes 190, required to maintain the cells at an optimal concentration suitable for therapeutic use. At least a portion of the cells may be stored under appropriate conditions, such as in a cell banking facility, for later therapeutic use or further research.

Syringes 190 are individually and machine-readably identified by an individual identification code, such as bar code 122. The individual identification codes, such as bar codes 122, are typically machine read by bar code readers.

At every suitable stage of the methodology described hereinabove, appropriate quality control measures are taken, using inter alia inputs from computerized optical inspection and automatic provision of samples, which may be tested automatically or manually. This quality control functionality is designated by reference numeral 194 and is seen to interface with interactive computer controller 100 and via interactive computer controller 100 with other system components, as described generally hereinbelow.

In accordance with an embodiment of the present invention, quality control functionality 194 employs optical inspection functionalities, such as functionalities designated by reference numerals 125 and 144 to provide visual indications of various process parameters.

Quality control functionality 194 also employs pipetting modules 130, 150 and 180 for providing quality control samples of materials at various suitable stages of the methodology. These quality control samples are typically automatically or semi automatically evaluated by conventional techniques, in parallel or off-line. Such techniques include, for example, sterility analysis, endotoxin analysis, gram stain analysis, mycoplasma analysis, flow cytometry analysis, tube formation assays, immunoassays, such as ELISA, and tissue culture, as for evaluating colony forming units (CFU).

Based on the optical inspection functionalities and the evaluation of the quality control samples, interactive computer controller 100 may vary process parameters and process flow as appropriate and may order that certain batches of cells or other materials be discarded. The interactive computer controller may also interface with software administration tools or historical databases, and vary the process parameters and flow based on inputs received therefrom. For example, a comprehensive historical database may be maintained that includes biological data (e.g., measurements relating to cell numbers and physiology for all cells processed by the system) and system data (e.g., incubator CO2 levels or quantities of added chemicals that produced a desired result). This database may subsequently be queried in real time, to see whether processing of a given set of cells is proceeding in a manner consistent with the processing of earlier sets of cells. Deviations are typically reported for operator review in real time or off-line, and may be used as a reason for discarding a set of cells. In an embodiment, the interactive computer controller 100 may provide warnings to supervisory personnel and displays of system and methodology parameters of interest.

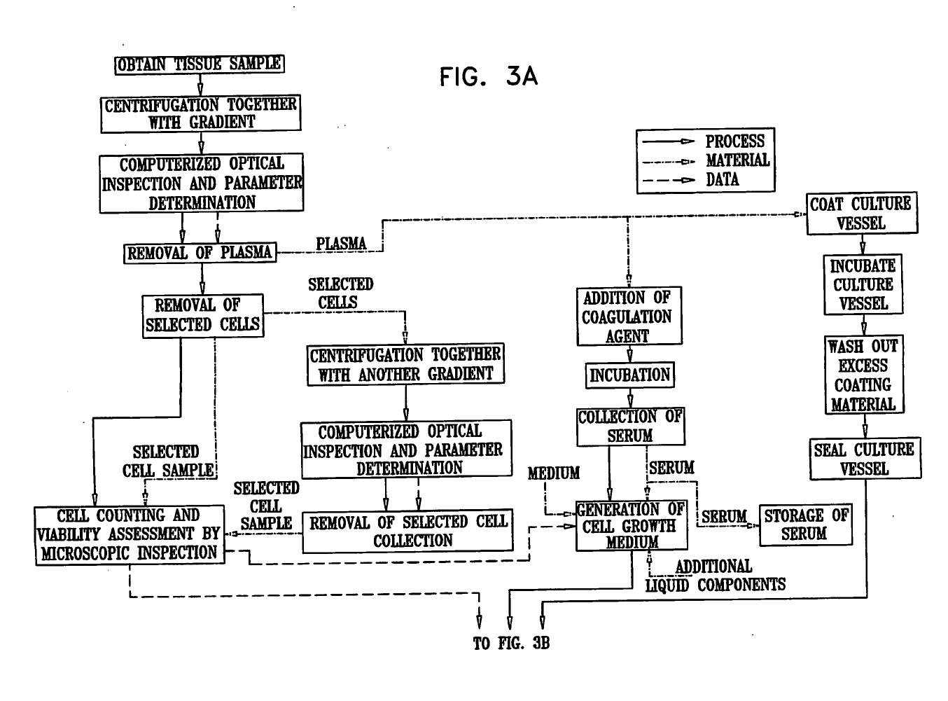

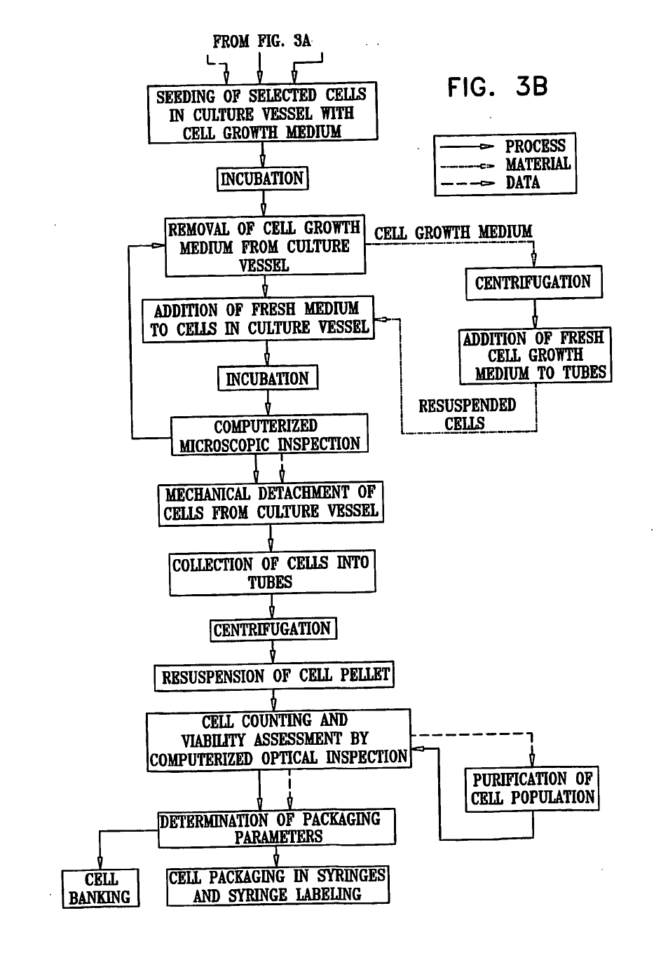

Reference is now made to FIGS. 3A and 3B which are detailed flow charts illustrating steps in the methodology illustrated generally in FIGS. 1 and 2. As described in FIG. 3A, the tissue sample 110 (FIG. 1) is mixed by gently flipping the tissue sample container such as blood storage bag 112 (FIG. 1) up and down, and then transferred into a suitable sterile vessel. Subsequently, and typically utilizing a pipetting module 130 (FIG. 1), the tissue sample is loaded onto a 1.077 g/ml density gradient 114 (FIG. 1), for example a Ficoll gradient such as Ficoll-Paque Plus™ which is commercially available from Amersham Biosciences, Uppsala, Sweden or Lymphoprep™ which is commercially available from Axis shield PoC AS, Oslo, Norway, prepared in a suitable number of tubes 116 (FIG. 1). Alternatively, the gradient may be prepared by the operator or obtained from any other suitable source.

It is appreciated that pipetting modules, such as pipetting module 130, 150 and 180, are employed in most of the liquid handling functions, such as aspiration, addition of liquid, dilution and suspension described hereinbelow.

The tubes 116 are then centrifuged in centrifuge module 120 (FIG. 1) together with the gradient 114, typically for approximately 20 minutes, at room temperature, at a suitable speed such as 1050 g and with no brake, and subjected to computerized optical inspection by optical inspection functionality 125 (FIG. 1) for the purpose of differentiating cells by the relative location of cell layers and the grey level or color of light reflected therefrom. Based on the outputs of optical inspection functionality 125, interactive computer controller 100 (FIG. 1) determines suitable parameters for separation of the resulting fractions.

Following centrifugation, most of the plasma 126 (FIG. 1) is collected from the upper layer of the centrifuged tubes 116 into empty tubes using pipetting module 130 and subsequent thereto, a fraction of selected cells 132 (FIG. 1), typically including peripheral blood mononuclear cells (PBMC), is removed from each of tubes 116 by pipetting module 130 and transferred into one or more new tube 136 (FIG. 1) which are typically prefilled with a suitable quantity of a suitable fluid such as Phosphate Buffered Saline (PBS) which is commercially available from Sigma-Aldrich Ltd. of St Louis, Mo., USA. It is appreciated that although one layer of selected cells is typically removed at any given time, multiple layers of selected cells may be sequentially removed. Typically, the volume in each of the tubes 136 is adjusted to a desired volume, typically 30 ml per tube. The tubes 136 are typically spun for approximately 15 minutes at a suitable speed such as 580 g, at room temperature, in the centrifuge module 120 and the supernatant is discarded based on an output of optical inspection functionality 125. The cell pellet is then mixed and re-suspended with a suitable quantity of liquid, typically with 1-5 ml of PBS. The contents of several tubes 136, typically four tubes, are subsequently combined into one tube.

For each layer of selected cells, microscopic inspection is carried out by microscopic inspection module 140 (FIG. 1) for cell counting and viability assessment. Cell counting is typically performed by pre-filling each of a suitable number of microtubes (not shown), with an appropriate quantity, typically 80 μl , of Trypan Blue (TB), which is commercially available from Sigma-Aldrich Ltd. of St Louis, Mo., USA. Subsequently, dilutions of a 1:5 ratio are prepared by transferring 20 μl of a cell sample to one Trypan Blue containing microtube, mixing gently by pipetting up and down and loading 10 μl of the diluted cells onto each of the 2 chambers of hemocytometer located under the microscope in microscopic inspection module 140.

Subsequently, clear (viable) and blue (dead) cells lying in the central 25 squares of each of the chambers are counted. If fewer than 10 cells are counted, the cells are counted again in a less diluted sample, typically a 1:2 dilution prepared by transferring 50 μl of the cell sample into 50 μl Trypan Blue. If more than 200 cells are counted, the cells are counted again in a more diluted sample, typically a 1:25 dilution prepared by transferring 20 μl of the previously prepared 1:5 dilution cell suspension to one Trypan Blue-containing microtube, and mixing gently by pipetting up and down employing a pipetting module which is similar to pipetting modules 130 and 150 (FIG. 1).

The cell number in each chamber is then calculated, typically according to the following equations:

No. of viable cells×10,000×Dilution factor=No. of viable cells/ml.

No. of dead cells×10,000×Dilution factor=No. of dead cells/ml.

% Dead cells=No. of Dead cells/(No. of Viable cells+No. of Dead cells)×100.

% of dead cells should not typically exceed 30%.

The average cell number is calculated based on the results of the above calculations, the final cell numbers are summarized, and yield of cells/ml blood is determined.

The cells in each layer may be further purified by one or more additional centrifugations employing centrifuge module 120, with a more selective gradient such as a Percoll™ gradient, including polyvinylpyrrolidone-coated silica colloids, which is typically available from Amersham Biosciences of Uppsala, Sweden, and which is suitable for selecting cells having a density less than 1.072 g/ml or less than 1.062 g/ml. As appropriate, other density gradients may be used, such as OptiPrep™ and Nycodenz™ gradients, which are commercially available from Axis shield PoC AS, of Oslo, Norway. In any such case, the centrifuged tubes are typically subjected to computerized optical inspection by optical inspection functionality 125 for the purpose of differentiating cells by the relative location of cell layers and the grey level or color of light reflected therefrom. The location of the desired layer may also be identified by comparison with another tube containing cellular density marker beads or another type of marker. Cells in layer(s) designated for removal are identified based on the locations of the marker beads in the other tube having corresponding densities.

Based on the outputs of optical inspection functionality 125, interactive computer controller 100 determines suitable parameters for separation of the resulting fractions. Subsequently, one or more collections of selected cells may be removed and subjected to further microscopic inspection by microscopic inspection module 140 for cell counting and viability assessment as described hereinabove.

The result of this stage is one or more collections of selected cells whose number and viability have been assessed.

At least some of the plasma 126 which was removed prior to removal of the selected cells is combined with a suitable quantity of a coagulation agent, such as CaCl₂, commercially available from American Pharmaceutical Partners Inc. of Schaumburg, Ill., USA, or any other suitable chemical or biological coagulation agent such as thromboplastin, thrombin agonist peptides or any other suitable coagulation agent, and incubated in incubator module 160 (FIG. 1), typically for half an hour to four hours at 37° C., thus producing a clot and serum. Following incubation, the serum is obtained inter alia by squeezing the generated clot, or in any other suitable method.

Subsequently, the serum is collected into a new tube, and combined with a medium, such as X-Vivo 15, commercially available from Cambrex Corporation of East Rutherford, N.J., USA, and additional liquid or soluble components which typically include at least some of erythropoietin (EPO), Insulin-like Growth Factor (IGF), basic-Fibroblast Growth Factor (b-FGF), Vascular Endothelial Growth Factor (VEGF), Heparin, Statin molecules, antidiabetic agents such as Rosiglitazone, molecules from the estrogen and progesterone families and combinations thereof, typically in concentrations in the range of 0.5 μg/ml-100 μg/ml, to produce a suitable cell growth medium. Typically, the serum comprises 1-20% of the resulting cell growth medium. Any surplus serum is typically stored and may be employed in other processes. As an alternative to using the serum for the production of cell growth medium, the serum may be stored at −20° C. until use.

It is a particular feature of some embodiments of the invention that the amount of cell growth medium produced is calculated based on the number of viable cells.

A suitable number of culture vessels 146 (FIG. 1) may be prepared by coating the interior thereof with the plasma 126 removed from the centrifuged tissue sample or alternatively with plasma from a different source, such as plasma commercially available from Chemicon of Temecula, Calif., USA. Alternatively, the coating may be any other suitable cell adhesion enhancer, such as a material manufactured from proteins derived from plasma other than from the patient, for example Fibronectin, commercially available from Chemicon of Temecula, Calif., USA. Alternative cell adhesion enhancers may include an antibody, a growth enhancing molecule such as collagen and other growth enhancing molecules. When coating the culture vessels 146 with plasma, each culture vessel is filled with a suitable amount of plasma, typically 2-5 ml, by a pipetting module such as pipetting module 130.

Alternatively, when coating the culture vessels 146 with fibronectin, a fibronectin solution in a suitable volume and at a suitable concentration, typically 50 ml of 25 μg/m1 fibronectin solution in PBS, is prepared, typically by adding 250 μl fibronectin 5 mg/ml to 50 ml PBS. Each of the culture vessels 146 is then filled with a suitable amount of fibronectin solution, typically 2-5 ml, by a pipetting module similar to pipetting module 130.

The culture vessels 146 are then incubated in incubator module 160, typically at 37° C. and for at least 30 min. Following incubation, the coating liquid is discarded by employing a pipetting module similar to pipetting module 130, and the culture vessels are washed twice with a suitable washing liquid such as PBS to flush out excess coating liquid. The culture vessels 146 are then allowed to dry, and are kept sealed at room temperature.

The number of culture vessels is calculated based on the calculated number of viable cells, and on the required concentration of cells in each culture vessel

Turning now to FIG. 3B, it is seen that a cell suspension containing a suitable number of the selected cells is split into an appropriate number of tubes, which are then spun in the centrifugation module 120 for approximately 15 minutes at room temperature and at an appropriate speed, typically 500 g, and the supernatant is discarded. The cell pellet is then gently mixed and re-suspended in cell growth medium to a suitable concentration, typically 5-50×10⁶ cells/ml, typically by a pipetting module 130.

Subsequently, the cells are seeded in each of the pre-coated culture vessels 146, typically at a concentration of 1-5×10⁶ cells/ml together with a suitable amount of cell growth medium, and each culture vessel 146 is incubated in incubator module 160, at 37° C. and in an environment having 5% CO₂.

During the incubation period, typically after a number of days, typically three, the cell growth medium and non-adherent cells contained therein are collected from every culture vessel 146 into a tube by employing a pipetting module such as pipetting module 150. Each culture vessel 146 is filled with an appropriate amount of fresh cell growth medium, typically 10 ml. The removed cell growth medium may optionally be centrifuged in centrifuge module 120, typically for approximately 10 minutes at 450 g, at room temperature, to collect non-adherent cells.

The cell pellet may be resuspended in an appropriate amount of fresh cell growth medium, typically 10 ml per culture vessel 146, and reintroduced into the culture vessels 146. Alternatively, the non-adherent cells may be removed and discarded and fresh cell growth medium not containing any cells added into the culture vessels 146. The culture vessels 146 are then subjected to further incubation. At any suitable time during incubation, a microscopic inspection, flow cytometry analysis or any other analytic evaluation method may take place in microscopic inspection module 170 (FIG. 1) or another analytic instrument and a decision may be made whether to continue incubation with the same or a different cell growth medium or to harvest the cells based on the output of microscopic inspection module 170.

It is appreciated that the type of cell growth medium used may be changed each time the cell growth medium is refreshed, for example, in order to further enhance the purity of the cell population. This is specifically important when directing or redirecting the differentiation of multipotent cells.

Typically, after one or more cell growth medium refreshments, typically within 30 days and typically after five days, the cells are harvested, typically by mechanical detachment thereof from the culture vessel 146. Alternatively, the cells may be detached from culture vessels 146 by chemical detachment, such as by use of trypsin.

Prior to mechanical detachment, the cell growth medium and non-adherent cells contained therein are collected from every culture vessel 146 into a tube 178 (FIG. 1) by employing a pipetting module such as pipetting module 150. Thereafter, the surface of each of the culture vessels 146 is carefully washed by pipetting with a suitable fluid, such as 10 ml cold PBS. Any remaining cells, which are suspended in the PBS washing liquid, are collected into tubes 178 containing the non-adherent cells previously collected. Additional liquid in an appropriate quantity, such as 5 ml cold PBS is added to the culture vessel 146, the adherent cells are detached from the culture vessel using gentle round movements with a cell scraper, and are subsequently collected and added to the tubes 178. Following the collection of cells, the culture vessel 146 is inspected by optical inspection module 170 in order to monitor the presence of cells in the vessel 146. Output of optical inspection module 170 is supplied to interactive computer controller 100 in order to determine whether further scraping is required. The culture vessels 146 are washed in an appropriate volume of liquid, typically 10 ml cold PBS to remove any remaining cells, and the washing fluid is added to the tubes 178.

The harvested cells are now suspended in a combination of PBS and cell growth medium, typically in a single test tube per culture vessel 146. Subsequently, the tubes are spun at suitable conditions, typically for 10 minutes at 450 g at room temperature, and the cell pellet is resuspended in an appropriate volume of a different liquid, typically a medium such as X-Vivo 15.

Computerized microscopic inspection is carried out in microscopic inspection module 140 for cell counting and viability assessment as described hereinabove. Suitable selection of cell types may take place at this stage to enhance the purity of the cell population. The purity of the cell population is optionally enhanced by use of CD133-conjugated magnetic beads (which are commercially available from Miltenyi Biotec GmbH of Bergisch Gladbach, Germany) or beads conjugated to a different marker.

A suitable amount of FcR Blocking Reagent, typically 100 μl,is typically added to 10⁸ total cells resuspended in a suitable amount of an appropriate fluid, such as 300 μl of buffer, to inhibit unspecific or Fc-receptor mediated binding of antibodies to non-target cells. The cells are then labeled by adding a suitable amount of CD133 MicroBeads, typically 100 μl, to a final volume, typically of 500 μl per 10⁸ total cells. The mixture is then incubated, typically for 30 minutes at 4-8° C.

Following incubation, the cells are washed by adding 10-20× the labeling volume of buffer and are centrifuged at appropriate conditions, typically at 300 g for 10 minutes. The supernatant is then removed, and the cell pellet is resuspended in a suitable amount of a suitable fluid, typically 500 μl buffer for up to 10⁸ total cells. The cells are now ready for separation on a magnetic bead column, thus enhancing the purity of the cell population.

A suitable magnetic bead column, such as an MS Column suitable for up to 10⁷ magnetically labeled cells or up to 2×10⁸ total cells or an LS Column suitable for up to 10⁸ magnetically labeled cells or up to 2×10⁹ total cells, is selected, and is placed in a magnetic field of a suitable Magnetic Cell Sorting Separator (MACS® Separator).

The column is prepared by rinsing it with an appropriate amount of buffer, and the cell suspension is then applied into the column. The non-labeled cells, typically the non-selected cells, are allowed to pass through the column, and the column is washed with an appropriate amount of buffer. Following the removal of non-labeled cells the column is removed from the magnetic field separator and is placed on a suitable collection tube. An appropriate amount of buffer is pipetted into the column, and the fraction of labeled cells is flushed out as by use of a plunger which is supplied with the column.

The magnetic separation step may be repeated as necessary, typically by applying eluted cells to a new prefilled positive selection column. The purified cells are spun at suitable conditions, typically for 10 minutes at 450 g at room temperature, and the cell pellet is resuspended in an appropriate volume of a different liquid, typically a medium such as X-Vivo 15. Computerized microscopic inspection is carried out in microscopic inspection module 140 for cell counting and viability assessment as described hereinabove.

Following cell counting, and based on cell count outputs, clinical requirements relating to cell concentration and injection volumes, packaging parameters are determined by interactive computer controller interactive computer controller 100. Based on the computerized calculations by interactive computer controller 100, the cells are suspended to an appropriate concentration and automatically placed in a suitable number of suitably sized syringes 190 (FIG. 1) or other delivery vessels. Alternatively or additionally, some or all of the cells may be resuspended to another appropriate concentration and stored in a cell banking facility for future therapeutic use or for further research and development.

Typically, automated packaging functionality comprises pipetting functionality, a tube or syringe holder, and/or an automatic arm that holds, shakes, rotates, and translates the tubes or syringes, as appropriate. In addition, the packaging functionality typically comprises an arm to place the tubes or syringes in a bubble wrap pack, seal the bubble wrap pack, label it with a computer-readable source of information, such as a barcode, an RFID, or a magnetic stripe, and, as appropriate, also place the pack in a cooling device for shipment (or in any other shipment packaging).

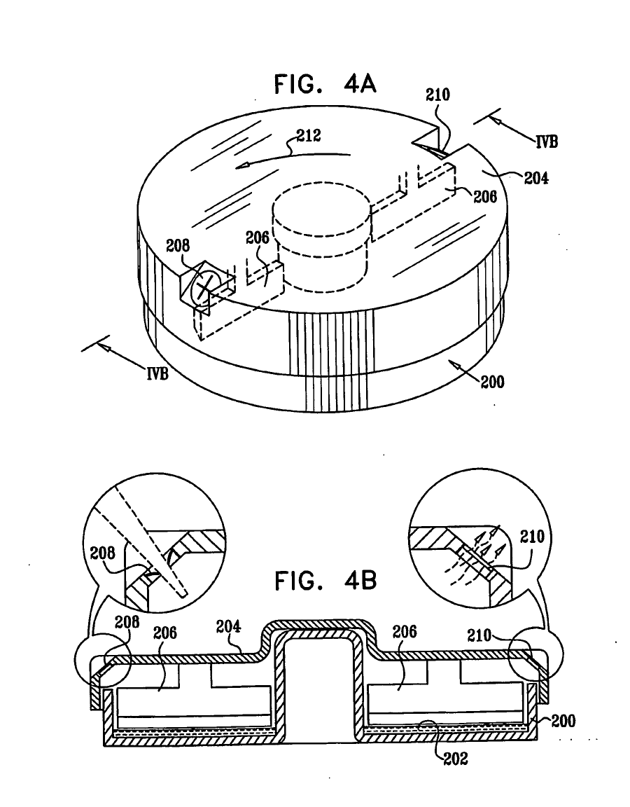

Reference is now made to FIGS. 4A and 4B, which are respective simplified pictorial and sectional illustrations of a self-scraping cell culture assembly constructed and operative in accordance with an embodiment of the present invention. The embodiment of FIGS. 4A and 4B may be employed in place of conventional culture vessels 146 illustrated in FIG. 1 to provide enhanced ease of cell growth and removal.

As seen in FIGS. 4A and 4B, the self-scraping cell culture assembly comprises a generally annular dish 200, typically having a raised center portion and defining a generally flat, ring shaped cell growth surface 202. A cover 204 is arranged for sealing engagement with annular dish 200 and is typically provided with one or more integrally formed scraper blades 206, which engage ring shaped cell growth surface 202. Cover 204 typically is formed with a septum cap 208 as well as a vented cap 210.

Rotation of cover 204 in a direction such as that illustrated by an arrow 212 relative to annular dish 200 provides scraping of cells from ring shaped cell growth surface 202. The cells, once thus detached from surface 202 may be removed from the dish 202 by pipetting through septum cap 208.

It will be appreciated by persons skilled in the art that the present invention is not limited to what has been particularly shown and described hereinabove. Rather the present invention includes both combinations and subcombinations of the features described hereinabove as well as modifications and variations which would occur to persons skilled in the art upon reading the foregoing description and which are not in the prior art.

1. The automated cell processing system according to claim 2, wherein
said automatic functionality modulates both said proportion of said cells of said at least one of said multiple cell types as compared with at least another of said multiple cell types and also modulates the absolute number of said cells of said at least one of said multiple cell types.

2. An automated cell processing system comprising:
functionality which receives at least one tissue containing a multiplicity of cells belonging to multiple cell types; and
automatic functionality including cell culturing which modulates the proportion of cells of at least one of said multiple cell types as compared with at least another of said multiple cell types.

3. (canceled)

4. An automated cell processing system according to claim 2, wherein the automatic functionality including packaging functionality for packaging at least one of said multiple cell types.

5. The automated cell processing system according to claim 2, wherein
said functionality which receives said at least one tissue comprises a first functionality which receives at least one tissue containing a first plurality of cell types having a first proportionality of numbers, and wherein said system further comprises:
at least one second functionality operative on at least some of said first plurality of cell types; and
adaptive control functionality operative to control the operation of said at least one second functionality as a function of at least some of said numbers.

6-7. (canceled)

8. The automated cell processing system according to claim 2, wherein:
a said functionality which receives said at least one tissue comprises a first functionality which receives at least one tissue containing a first plurality of cell types having a first proportionality of numbers, and wherein said system further comprises:
at least one second functionality operative with respect to at least some of said first plurality of cell types to at least one of add, mix or remove at least one material;
monitoring functionality operative to monitor said at least some of said first plurality of cell types; and
adaptive control functionality operative to control the operation of said at least one second functionality in response to at least one output of said monitoring functionality.

9. (canceled)

10. The automatic cell processing system according to claim 2, wherein said system further comprises an automated cell bank generating system comprising:
storage functionality for storing said at least one of said multiple cell types.

11-12. (canceled)

13. The automatic cell processing system according to claim 2, wherein
said functionality which receives said at least one tissue comprises a first functionality which receives said at least one tissue containing a first plurality of cell types having a first proportionality of numbers, and wherein said system further comprises:
at least one second functionality operative on at least some of said first plurality of cell types;
monitoring functionality operative to monitor differentiation of said at least some of said first plurality of cell types; and
adaptive control functionality operative to control the operation of said at least one second functionality in response to at least one output of said monitoring functionality.

14-15. (canceled)

16. The automatic cell processing system according to claim 2, wherein said system further comprises an automated autologous cell therapy system comprising
autologous implantation functionality for supplying said at least one of said multiple cell types to a source of said at least one tissue.

17-21. (canceled)

22. The automatic cell processing system according to claim 2, wherein said system further comprises an automated non-autologous cell therapy system comprising:
non-autologous implantation functionality for supplying said at least one of said multiple cell types to a recipient other than a source of said at least one tissue.

23-28. (canceled)

29. The automated system according to claim 2, wherein said automatic functionality increases said proportion of cells.

30. The automated system according to claim 2, wherein said automatic functionality increases the absolute number of said cells of said at least one of said multiple cell types.

31. The automated system according to claim 2, wherein said automatic functionality increases said proportion of cells and the absolute number of said cells of said at least one of said multiple cell types.

32-49. (canceled)

50. The automatic cell processing system according to claim 2, further comprising a self-scraping cell culture assembly for receiving said multiplicity of cells, the assembly comprising:
a generally annular dish defining a generally flat, circularly-shaped cell growth surface;
a cover arranged for sealing engagement with said annular dish; and
at least one scraper blade mechanically associated with said cover, whereby rotation of said cover relative to said dish provides scraping of said cells from said circularly-shaped cell growth surface.

51. The self-scraping cell culture assembly according to claim 50, wherein said cover is formed with at least one component selected from the group consisting of: a septum element operative for insertion of a pipetting device into said annular dish, and a vent.

52. The automated cell processing system according to claim 2, wherein
said functionality which receives said at least one tissue comprises a first functionality which receives a culture comprising at least one tissue containing a first plurality of cell types, and wherein said system further comprises:
at least one second functionality operative on at least some of said first plurality of cell types; and
adaptive control functionality operative to control the operation of said at least one second functionality as a function of at least one parameter of said culture.

53-63. (canceled)

64. The method for automated cell processing according to claim 89, wherein:
modulating said proportion of cells comprises automatically increasing both the proportion of said cells of said at least one of said multiple cell types as compared with said at least another of said multiple cell types and the absolute number of cells of said at least one of said multiple cell types.

65. The method for automated cell processing according to claim 89, wherein
modulating said proportion of cells comprises increasing the proportion of said cells of said at least one of said multiple cell types as compared with said at least another of said multiple cell types.

66. The method for automated cell processing according to claim 89, wherein
receiving said at least one tissue comprises receiving at least one tissue containing a first plurality of cell types having a first proportionality of numbers, and wherein said method further comprises:
operating on at least some of said first plurality of cell types; and
controlling said operating as a function of at least some of said numbers.

67. The method for automated cell processing according to claim 89, wherein
receiving said at least one tissue comprises receiving at least one tissue containing a first plurality of cell types having a first proportionality of numbers, and wherein said method further comprises:
operating on a second plurality of cell types being at least some of said first plurality of cell types and having a second proportionality of numbers which varies over time;
monitoring variations in said numbers of said second plurality of cell types; and
controlling said operating as a function of at least some of said variations in said numbers of said second plurality of cell types.

68. The method for automated cell processing according to claim 89, wherein
receiving said at least one tissue comprises receiving at least one tissue containing a first plurality of cell types having a first proportionality of numbers, and wherein said method further comprises:
operating on at least some of said first plurality of cell types;
monitoring differentiation of said at least some of said first plurality of cell types; and
controlling said operating in response to at least one output of said monitoring functionality.

69-87. (canceled)

88. The method for automated cell processing according to claim 89, wherein
automatically culturing said cells comprises automatically modulating both the proportion of said cells of said at least one of said multiple cell types as compared with said at least another of said multiple cell types and the absolute number of cells of said at least one of said multiple cell types.

89. A method for automated cell processing comprising:
receiving at least one tissue containing a multiplicity of cells belonging to multiple cell types; and
automatically culturing said cells thereby modulating the proportion of cells of at least one of said multiple cell types as compared with at least another of said multiple cell types.

90. (canceled)

91. The method for automated cell processing according to claim 89, further comprising generating a cell bank by
storing said at least one of said multiple cell types.

92. (canceled)

93. The method for automated cell processing according to claim 89, further comprising:
following said culturing, facilitating autologous cell therapy by supplying said at least one of said multiple cell types to a source of said at least one tissue.

94. (canceled)

95. The method for automated cell processing according to claim 89, further comprising:
following said culturing, facilitating non-autologous cell therapy by supplying said at least one of said multiple cell types to a recipient other than a source of said at least one tissue.

96. A self-scraping cell culture assembly comprising:
a generally annular dish defining a generally flat, circularly-shaped cell growth surface;
a cover arranged for sealing engagement with said annular dish; and
at least one scraper blade mechanically associated with said cover, whereby rotation of said cover relative to said dish provides scraping of cells from said circularly-shaped cell growth surface.

97. A self-scraping cell culture assembly according to claim 50 and wherein said cover is formed with at least one of a septum element operative for insertion of a pipetting device into said annular dish, and a vent.
